(19) United States
(12) Patent Application Publication
(10) Pub. No.: US 2024/0168000 A1
(43) Pub. Date: May 23, 2024

(54) SPECTRAL IMAGING FOR MATERIAL CHARACTERIZATION AND CONTROL OF SYSTEMS AND METHODS FOR PROCESSING EARTHEN MATERIALS

(71) Applicant: MOTION METRICS INTERNATIONAL CORP., Vancouver (CA)

(72) Inventors: Obada Alhumsi, Vancouver (CA); Sophia Alexandra Helen Piche, Vancouver (CA); Peter Shang Yu Hsieh, Port Moody (CA); Mohammad Sameti, Coquitlam (CA); Saeed Karimifard, Vancouver (CA); Thomas C. Chudy, Coquitlam (CA)

(73) Assignee: MOTION METRICS INTERNATIONAL CORP., Vancouver (CA)

(21) Appl. No.: 18/516,847

(22) Filed: Nov. 21, 2023

Related U.S. Application Data
(60) Provisional application No. 63/427,085, filed on Nov. 21, 2022.

(51) Int. Cl. G01N 33/24; G01N 1/28; G01N 21/31

(52) U.S. Cl. CPC G01N 33/24; G01N 1/286; G01N 21/31; G01N 2201/12761; G01N 2201/1296

(57) ABSTRACT

Spectral imaging systems are used to gather spectral image data on earthen material moving within an earthen material processing system, such as a mineral processing system or cement plant. Machine learning models such as 3D convolutional neural networks may be utilized to process the spectral image data to determine or classify one or more characteristics of the earthen material, such as ore grade, mineral alteration(s), moisture content, lithology and/or mineralogy. Such earthen material characteristics, or classifications thereof, may then be utilized to automatically control one or more operational characteristics of the earthen material processing system, such as rotational speed of milling equipment or flow rates of water or chemicals added to milling equipment or mineral concentration systems.

### RELATED APPLICATIONS

This application claims priority to U.S. Provisional Patent Application No. 63/427,085 filed Nov. 21, 2022 entitled “HYPERSPECTRAL IMAGING FOR MATERIAL CHARACTERIZATION AND CONTROL OF SYSTEMS AND METHODS FOR PROCESSING EARTHEN MATERIALS,” which is incorporated by reference in its entirety herein and made a part hereof.

### TECHNICAL FIELD

This disclosure relates to systems and methods for material characterization using spectral imaging (spectral imaging) and image analysis techniques. More particularly, this disclosure relates to the use of spectral imaging systems and methods, in-line with an earthen material conveyor or other dynamic portion of a system for processing earthen materials, for controlling the earthen material processing system and related processing operations. Such earthen material characterization systems and methods may be particularly useful in mineral processing systems for copper, iron, and other metals and systems and methods for making cement, concrete, and adhesives.

### BACKGROUND

Mineral processing involves systems for comminution of earthen material, which may include ore or other mineral deposits, and for concentration or liberation of minerals from the earthen material. Such mineral processing systems are generally located at or near a mine site due to the high cost associated with transporting unprocessed earthen material that has a relatively low mineral content. Comminution is the reduction in size of earthen material into particles through multiple stages of crushing, grinding, and sizing or classification (sorting). Concentration is the separation of the desired mineral(s) from the worthless or unwanted material called gangue that surrounds the mineral(s) in the earthen material deposits. Concentration may involve mechanical, physiochemical, optical, electrostatic, and/or magnetic separation of mineral particles from gangue particles; and in some cases, may involve chemical liberation (e.g., solvent extraction) of the mineral(s) from the particles and subsequent stripping of the mineral(s) from solution via electrowinning or precipitation. Concentrated mineral is then transported to a refinery or smelter for further processing into a high-purity metal or other marketable form of the mineral.

Notwithstanding efforts to extract earthen material of consistently high ore grade, the actual ore grade, mineralogy, lithology, and other characteristics of earthen material can vary widely during the excavation process. Consequently, the characteristics of earthen material conveyed to and through a mineral processing facility can fluctuate unpredictably, which can impact comminution and/or concentration operations, degrading the quantity or quality of their output, increasing energy usage, and/or increasing environmental impacts of the processes or of the tailings exiting the mineral processing facility.

### SUMMARY OF THE DISCLOSURE

Systems and methods are disclosed for characterizing earthen material in an earthen material processing system, such as a mineral processing system or cement plant, and for making a recommendation for and/or automatically controlling at least one controllable operational parameter of the earthen material processing system with the exclusion of transport systems. Such systems and methods may include a spectral imager positioned in view of earthen material moving within the earthen material processing system, wherein the spectral imager configured to acquire spectral image data of a spatial scene of the earthen material. A processor in communication with the spectral imager may be programmed with a machine learned model that is configured to process the spectral image data to determine an earthen material characteristic of the earthen material based on the spectral image data. The processor may output a signal based on the earthen material characteristic determined by the machine learned model, which signal is communicated to the earthen material processing system for making a recommendation for and/or automatically adjusting the operational parameter of the earthen material processing system in response to the signal.

In some systems, the spectral imager is configured to acquire the spectral image data while the earthen material is moving, for example by positioning the spectral imager over a conveyor of the earthen material processing system. The spectral imager may capture successive lines, batches, sets, and/or patterns of spectral data, which may be aggregated by the spectral imager or the processor to form the spectral image data. The spectral imaginer may be a hyperspectral camera by HySpex Baldur S-384 N having a spectral range of 960-2500 nm and 288 spectral bands, but other spectral imagers may be used, e.g., with less bands. The conveyor may deliver a flow of earthen material to a comminution system of the earthen material processing system, and the controllable operational parameter automatically adjusted in response to the signal may include an operational parameter of the comminution system, such as one or more of a grinding media volume of the mill, a rotational speed of the mill, a flow rate of water delivered to the mill, and a dewatering rate of the mill. In some systems and methods, the spectral imager may be located within a reclaim tunnel of the earthen material processing system. The system may include one or more illumination sources positioned and oriented to direct illumination toward the earthen material for reflection by the earthen material to the spectral imager, to provide consistent high-intensity illumination in the visible spectrum, near infrared (IR) spectrum, and short wavelength IR spectrum.

Earthen material characteristics that may be determined from by the machine learned model from spectral image data may include ore grade, mineral alteration, moisture content, lithology and/or mineralogy; and one or more of such earthen material characteristics may be utilized to making a recommendation for and/or automatically control one or more operational characteristics of the earthen material processing system.

In some systems and methods, the earthen material processing system may include a mineral processing system including a comminution system and a concentration system, and the spectral imager may be located before or within the comminution system to gather spectral image data from which the machine learned model classifies a mineral alteration of the earthen material. Such systems and methods may making a recommendation for and/or automatically adjust a rate of addition of a reagent in the concentration system in response to the classification of the mineral alteration, wherein the reagent is reactive with one or more desirable minerals in the earthen material as part of a mineral concentration process such as froth floatation.

The machine learned model of systems and methods according to the present disclosure may include a convolutional neural network (CNN), such as a 3D CNN and/or 2D CNN. In some systems and methods disclosed herein, the processor is further programmed to preprocess the spectral image data to perform radiometric or geometric corrections, or other data manipulation, prior to processing by the machine learned model. In some systems and methods consistent with the present disclosure, the processor may be programmed to perform dimensionality reduction on the spectral image data prior to processing by the machine learned model.

Also disclosed are a methods of operating an earthen material processing system, which may involve acquiring a spectral image of earthen material moving within the earthen material processing system; processing spectral image data of the spectral image via a machine learned model operating on a data processor to determine an earthen material characteristic of the earthen material; outputting a signal based on the earthen material characteristic determined by the machine learning model; and making a recommendation for and/or automatically adjusting an operational parameter of the earthen material processing system in response to the signal.

Additional aspects and advantages of the disclosure will be apparent from the following detailed description of preferred examples, which proceeds with reference to the accompanying drawings.

### BRIEF DESCRIPTION OF THE DRAWINGS

FIG. 1 is a block diagram of the basic equipment and operations of an example comminution facility in accordance with the prior art.

FIG. 2 is a pictorial overview of a mineral processing facility utilizing spectral imaging systems and methods according to the present disclosure.

FIG. 2A is a schematic block diagram of the mineral processing facility of FIG. 2.

FIG. 3 is a schematic diagram of a cement plant utilizing spectral imaging systems and methods according to the present disclosure.

FIG. 4 is an isometric view of a spectral imaging camera and illumination source system mounted over a conveyor of an earthen material processing facility.

FIG. 5 is a longitudinal cross section view of the spectral imaging camera, illumination source system, and conveyor of FIG. 4.

FIG. 6 is a lateral cross section view of the spectral imaging camera and illumination source system of FIG. 4 taken along line 6-6 in FIG. 5.

FIG. 7 is an enlarged cross-sectional view of the spectral imaging camera of FIG. 6.

FIG. 8 is an isometric view of a variant of the spectral imaging camera and illumination source system of FIG. 4 with a cabinet and shrouds of the system omitted to show detail of the support frame and a modified arrangement of the illumination source system and illustrating a field of view of the spectral imaging camera.

FIG. 9 is a schematic block diagram of an earthen material characterization system utilizing spectral imaging systems and methods according to the present disclosure.

FIG. 10 is a block diagram of an exemplary computing environment suitable for use in implementing examples of the present disclosure.

FIG. 11 is a method for training a machine learning model to classify an ore grade characteristic of earthen material in order to control one or more processing parameters.

### DETAILED DESCRIPTION OF PREFERRED EXAMPLES

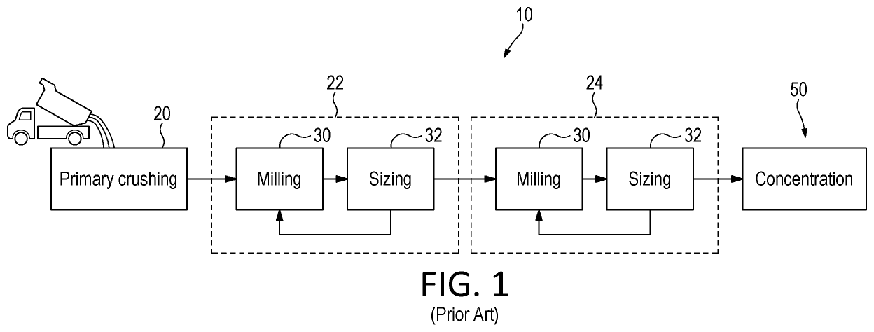

In a copper mining operation, a single comminution facility may process tens of thousands of metric tons of earthen material per day with numerous sequential processing operations running continuously in tandem. With reference to FIG. 1, earthen material that has been excavated or otherwise extracted from the mine is delivered to a primary crushing system 20, such as a jaw crusher or gyratory crusher, which reduces the gross size of larger pieces of earthen material to between about 6 cm and 30 cm, allowing further transport via conveyor belts or other dry material conveyor systems. The crushed earthen material is then transported via a conveyor to one or more secondary mill circuits 22 and then to one or more tertiary mill circuits 24, which further crush, grind, or otherwise mill the earthen material into progressively smaller particles. Depending upon the mine, the output of the final stage of the mill circuits 22, 24 is an extraction process that may include gravity and flotation, or redirected to ore extraction via heap leaching, or to a stockpile. For a gravity and flotation process, the output is typically a slurry that is delivered to a concentration facility or system for the separation or liberation of the desired mineral(s) from the gangue.

Each mill circuit 22, 24 typically may include a unit of milling equipment 30 for reducing pieces of earthen material into smaller pieces or particles, followed by a sizing operation 32 that passes smaller particles of earthen material to the next stage (e.g., a downstream mill circuit) of the comminution facility and typically returns larger particles back to the milling equipment of the particular mill circuit for further crushing and/or grinding into particles of the desired size. The milling equipment 30 of each mill circuit 22, 24 may include any of various types of mills for crushing or grinding earthen material, such as gyratory crushers, cone crushers, impact crushers, jaw crushers, high pressure grinding rolls (HPGRs), autogenous mills, semi-autogenous (SAG) mills, ball mills, cone mills, vertical regrind mills, pebble mills, or hammer mills. Water is added to some of these types of mills, and in some cases the water and milled particles are output in the form of a slurry that is transported to subsequent operations utilizing pumps, as is well known in the art. Sizing or classifying operations 32 at each mill circuit may involve screens and/or hydrocyclones which separate particles based on their relative size and/or specific gravity.

The final sizing or classifying operation 32 of the last mill circuit 24 outputs fine particles that are delivered to the concentration facility 50, which may include any of a variety of different systems for concentrating the mineral-rich particles, such as froth floatation, gravity separation, magnetic separation, heap leaching, stock piling, or electrostatic separation. The type of concentration process and the equipment utilized in the concentration facility 50 will depend on the type of minerals being processed. in one example, the concentrated mineral particles may then be dewatered through thickening and/or filtration and dried in a drier before being transported to a refinery or smelter. The concentration facility may involve chemical extraction processes instead or in addition to mineral particle concentration processes, resulting in one or more solutions from which one or more minerals can be stripped via electrowinning, heap leach, or precipitation.

In one example, ore extraction can be done via heap leaching. Heap leaching entails in principle the irrigation of earthen material by a lixiviant fluid which is dissolving the metal-bearing mineral phases and transporting the metal in a solution (pregnant solution) which is collected at the bottom of the heap and transported through channels and pipes to a processing plant where the metal is recovered through solvent extraction (SX) and electrowinning. The earthen material is placed in lifts on a slightly sloped surface lined with an impermeable geomembrane (leach pad) by either direct truck dump or mobile conveyor systems also referred to as (grass) hoppers. The earthen material can be crushed prior to placement on a leach pad or come directly from the mine face (run-of-mine). The leach pads can be in morphologically flat areas or fill out valleys. Individual lifts range in height from 6-8 meters and the overall height of a leach heap can reach up to 200 meters. The earthen material placed on such leach pads may undergo some pre-treatment including pelletization, acid-curing, and/or mixing with lime depending on its natural properties. Depending on the exact leaching process, the addition of bacterial colonies that feast on sulphides is an option that facilitates leaching of sulphide-rich earthen material. In some examples, systems for forced aeration may be installed at the bottom of the heap. The leaching process efficiency, leaching rate, and overall metal recovery depend on the characteristics of the earthen material such as mineralogical composition which includes the copper-bearing mineral species as well as the gangue (barren material hosting the copper minerals) composition, and particle size. Common issues encountered in heap leach operations include channelization or pooling of the lixiviant due to poor permeability and porosity of the earthen material, increased acid consumption due to high reactivity of the earthen material attributed to acid-consuming gangue minerals, poor recoveries due to presence of insoluble copper minerals, and/or death of bacterial colonies as a result of certain elements present in parts of the earthen material.

The characteristics of the earthen material to be processed are established during metallurgical testing at a project development stage. In a mineral processing facility, the ore grade, mineralogy, and other characteristics of the earthen material are conventionally determined by testing samples of earthen material offline using laboratory analysis techniques, such as X-ray fluorescence (XRF) spectrometry, X-ray diffraction (XRD) (also known as X-ray crystallography), inductively coupled plasma mass spectrometry (ICP-MS), and other analytical methods. And such laboratory test results may be utilized by mine personnel to make changes in the mix of earthen material being fed to the mineral processing facility (e.g., by drawing from multiple stockpiles having different characteristics), or to change operational parameters of equipment or process steps in the mineral processing facility. But because the characteristics of earthen material transported to and through the mineral processing facility are constantly changing, such laboratory analysis techniques are suboptimal. It has been proposed to utilize spectral imaging systems to characterize the ore grade, mineralogy, and lithology of earthen material in-situ or in laboratory settings. In a heap leach facility, the test work is completed on composites representing the average composition of the earthen material as well as some deviation from the average composition, and it focuses on aspects such as crush size sensitivity, ore grade, percolation, and leach kinetics. The inevitable limitations of the metallurgical test work relate to the sample representivity, duration of leach tests, and volume of material used in leach tests. As a result, heap leach extraction is relatively lower and extraction rates are much longer than initially established by metallurgical testing or conventional ore processing routes. As a result of the slow extraction rates, detection of any issues is drastically delayed, and corrective measures are difficult to apply once a heap is formed and irrigated.

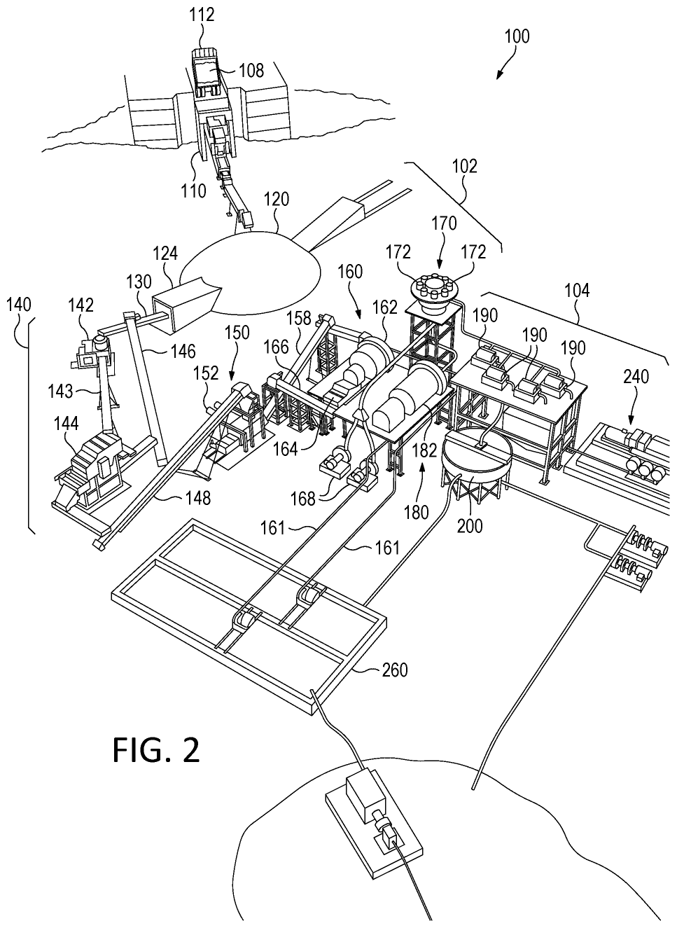

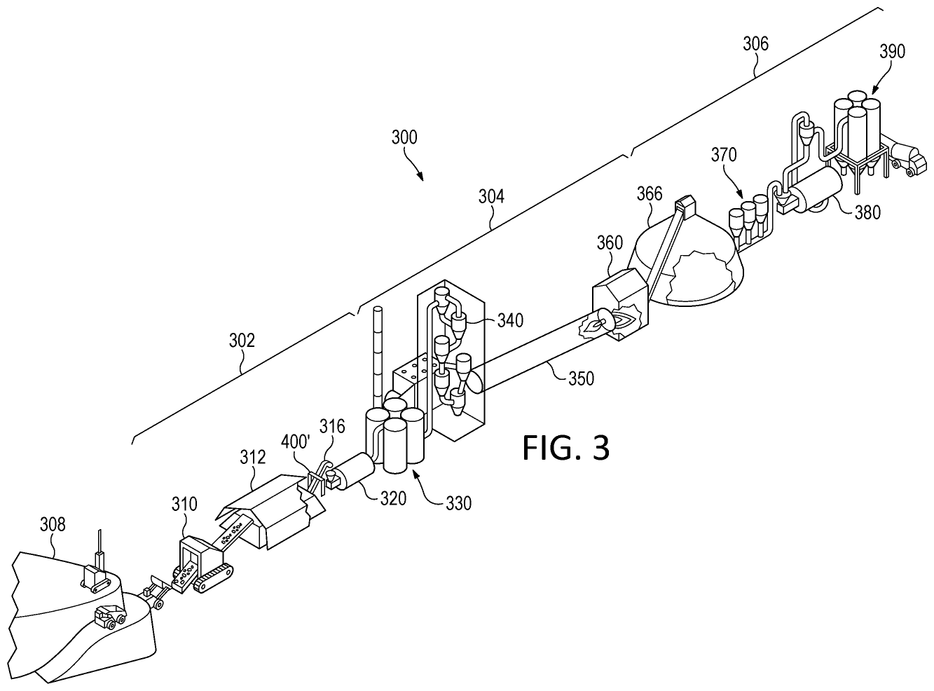

With reference to FIGS. 2 and 2A, the illustrated mineral processing facility 100 includes a comminution system 102 (also referred to as a comminution facility or comminution equipment) and a concentration system 104 (also referred to as a concentration facility or concentration equipment), but other configurations are possible. Raw earthen material 108 from a mine (not illustrated), such as an open pit mine or underground mine, is delivered to a primary crushing system 110 of the comminution system 102 by a dump truck 112 or other means of transport. The primary crushing system 110 may be a jaw crusher, gyratory crusher, or any other means of reducing the size of large pieces of earthen material. As illustrated in FIG. 2, the output of the primary crushing system 110 is deposited on a stockpile 120 over a reclaim tunnel 124 that houses part of a feed conveyor 130 of the comminution system 102. A spectral imaging system 400 (FIG. 2A), which is described below in detail with reference to FIGS. 4-8, may desirably be located within the reclaim tunnel 124 and mounted over the feed conveyor 130. One or more spectral imaging systems may optionally be located over other conveyors of the comminution system 102 downstream from the feed conveyor, either instead of or in addition to the spectral imaging system 400 located within reclaim tunnel 124. The spectral imaging system 400 may include a processor 1000, which may be located inside or outside of reclaim tunnel 124, may be connected to a network 1004 in communication with control systems and/or various processing equipment included in the mineral processing facility 100, and may optionally be connected to the Internet 1007, as further described below.

The feed conveyor 130 delivers earthen material from the stockpile to a first mill circuit 140 of the comminution system 102 including a secondary crushing machine 142, such as a cone crusher or other dry crushing system, which performs a dry milling operation. The secondary crushing machine 142 outputs crushed earthen material product via conveyor 143 to a sizing system of the first mill circuit 140, such as a screen 144 having uniform apertures that pass particles that are smaller than a desired size. Larger particles that do not pass through screen 144 are returned to the secondary crushing machine 142 via a return conveyor 146 of the first mill circuit 140, and smaller particles that pass through screen 144 are transported by a first transfer conveyor 148 to a second mill circuit 150 of comminution system 102.

The second mill circuit 150 may include a third crushing and/or grinding machine, such as a high pressure grinding rolls (HPGR) machine 152, for performing a dry milling operation. In the illustrated example, the second mill circuit 150 does not include a sizing system for returning larger particles because the output of the HPGR machine 152 is sufficiently uniform to eliminate the need for sizing or classification. The output of the HPGR machine 152 is transported by a second transfer conveyor 158 to a third mill circuit 160 of the comminution system 102. In some examples, the second mill circuit 150 may include a second sizing or classifying operation 154 (FIG. 2A) that returns larger particles to the HPGR machine 152 (or other milling equipment used in place of HPGR machine 152) and passes smaller particles to the third mill circuit 160 via the second transfer conveyor 158.

The third mill circuit 160 may involve a wet milling operation performed by a SAG mill 162 and a third sizing system having a screen 164 that catches larger particles and returns them to the SAG mill 162 via a second return conveyor 166 and transfer conveyor 158. Process water 161 is added to the SAG mill 162 and metered by a control system (not illustrated) that monitors the input process water of the SAG mill 162 and other parameters. The control system may be dedicated to the SAG mill 162 or centralized, for controlling various mill circuits and equipment of the comminution system 102. A slurry of process water and smaller particles that pass through the screen 164 is pumped via one or more pumps 168 to a classification system 170, which may include one or more hydrocyclones 172 that separate particles based on size and specific gravity. Larger particles exiting the hydrocyclones 172 from a lower spigot thereof are transported via piping to a fourth mill circuit 180 including a ball mill 182. Process water is added to ball mill 182 via a control system (not shown), which may be dedicated to the ball mill 182 or part of a centralized control system. The output of the ball mill 182 is a slurry that is pumped via one or more of the pumps 168 to classification system 170. A slurry of smaller particles exiting classification system 170 via an overflow port are transported via pipes to concentration system 104.

Concentration system 104 may include a set of froth floatation machines 190 and a tailings thickener 200. Concentration system 104 may also include chemical extraction operations for extracting desirable trace minerals before or after froth floatation and may further include other concentration steps or equipment. In operation, the overflow slurry from the classification system 170 flows into a tank of one or more of the froth floatation machines 190 and a reagent is introduced that binds with copper particles and causes them to become hydrophobic. Air bubbles are injected into the bottom of the tanks of the froth floatation machines 190 and carry the hydrophobic particles upwardly to the surface of the liquid in the tank. A concentrate of froth with the copper-rich particles is collected from the froth floatation machines 190 and transferred to a drying system 240 that produces dried copper concentrate particles, ready for shipment to a refinery or smelter. Subsets of the froth floatation machines 190 may be arranged in series, so that successive froth floatation operations are performed to extract additional copper concentrate. Process waste from the froth floatation machines 190 is transferred to the tailings thickener 200 where process water is recovered for treatment and the tailings are thickened before being output to a tailings pond 260. In some examples, a chemical extraction system 280 (FIG. 2A) may be utilized to extract chemical reagents and additional minerals (such as secondary or trace minerals included in the earthen material 108) from the process waste.

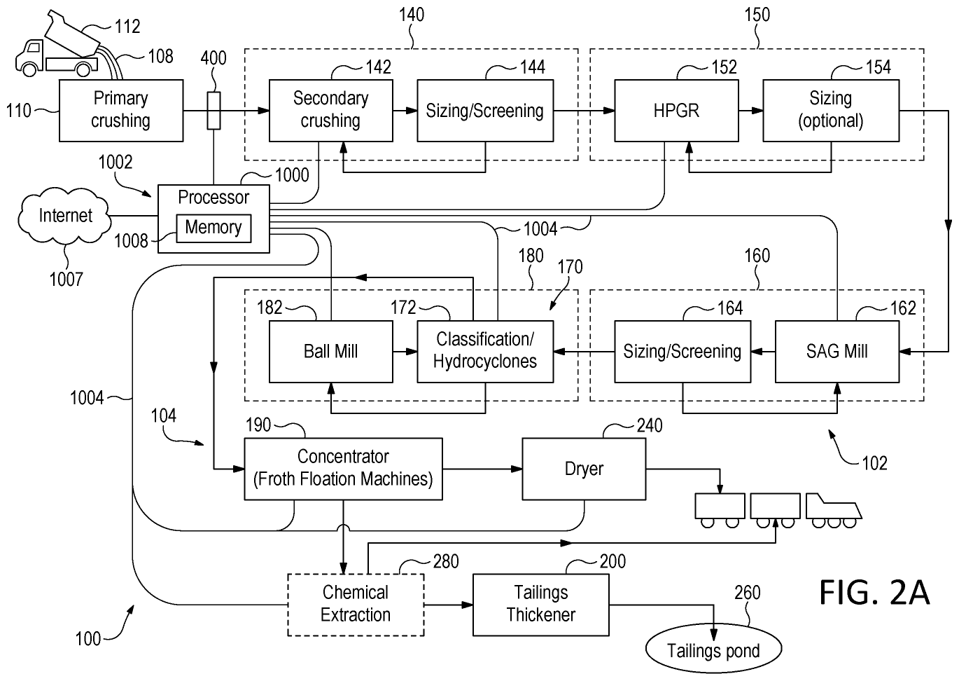

The foregoing description of mineral processing facility 100 is just one example of an arrangement of comminution systems 102 and particle concentration system 104 for copper sulfide earthen material. Depending on the type of earthen material being processed, mineral processing systems may include different comminution equipment and different concentration systems, any of which may benefit from control inputs provided by in-line categorization of feed earthen material by spectral imaging, as is further described below. For example, concentration systems may include other types of physiochemical concentration systems (different from froth floatation machines 190) and/or mechanical, electro-mechanical, electrochemical, and/or magnetic concentration systems. Furthermore, in-line spectral imaging systems according to the present disclosure may be utilized with other types of earthen material processing and handling equipment. Other examples of earthen material processing systems in which spectral imaging systems according to the present disclosure may be utilized include cement plants, concrete plants, and adhesives manufacturing facilities. An example of a cement plant utilizing spectral imaging is described below with reference to FIG. 3. Still other types of material processing facilities that may benefit from spectral imaging systems according to the present disclosure may include systems for use in processing agricultural materials, systems for material processing and/or material handling in manufacturing environments, and systems for use in refineries. In such facilities, information about the characteristics of the material derived through the use of in-line spectral imaging and analysis techniques can be used as control inputs to the control systems that establish and/or control the operating parameters of equipment and processes of such material processing facilities. Additional examples and detail of such material characteristics, control inputs, and operating parameters are described below.

As used herein, the term “in-line” refers to and means the utilization of spectral imaging according to the present disclosure in a production line or production environment in which or through which the subject imaged material to be analyzed moves, either continuously or intermittently, and should not be interpreted as limited to systems, configurations, or production environments having a linear arrangement or linear movement path or to only systems in which the material is in motion at the instant it is imaged. In-line spectral imaging systems also need not have all related processing equipment or methods present or performed in the production environment. For example, in systems with in-line imaging, some data processing and image analysis may be performed outside of the production environment, although it may generally be desirable to perform such processing and analysis immediately after imaging or in near real-time for facilitating timely control of material processing equipment downstream or later in the process from where or when imaging occurs. Other non in-line spectral imaging systems may be used.

FIG. 3 illustrates a cement manufacturing plant 300 in which spectral imaging systems and methods may be utilized according to the present disclosure. The cement manufacturing plant 300 may include a comminution system 302, a meal proportioning and burning system 304 and a finishing system 306. Cement manufacturing plant 300 may desirably be located in proximity to a quarry 308 from which raw earthen materials, such as limestone, are mined or excavated and then transported to a primary crushing device 310 of the comminution system 302. After crushing, the crushed raw earthen materials are conveyed to one or more stockpiles 312. One or more feed conveyors 316 transport crushed earthen material(s) from stockpile(s) 312 to one or more grinding mills 320, which grind and homogenize the earthen material(s) into fine particles. The grinding mills 320 may include one or more of various types of mills and/or an HPGR machine. The ground limestone particles are mixed with proportions of sand and clay at proportioning station 330 of the meal proportioning and burning system 304, then optionally further ground into meal by a meal grinding mill (not illustrated). The meal is then transferred to a preheating tower 340 before being fed into a rotary kiln 350 of the meal proportioning and burning system 304. The kiln 350 converts the meal to clinker which is output from kiln 350 to a clinker cooler 360 and then to clinker storage 366. The clinker is then blended with gypsum, and optionally other additives, at a blending station 370 of the finishing system 306 and then ground in a finish grinding mill 380 thereof to form cement that is then stored in silos 390 where it awaits shipment to a concrete batching plant (not shown) or for on-site mixing with aggregate and additives to form concrete. Conveyors, such as feed conveyor 316 and others not illustrated, may be provided between the various stations or processing equipment of cement manufacturing plant 300.

In one example, a spectral imaging system 400′ may desirably be located within a reclaim tunnel and mounted over the feed conveyor 316. One or more spectral imaging systems may optionally be located over other conveyors of the cement manufacturing plant 300 downstream from the feed conveyor 316, either instead of or in addition to the spectral imaging system 400′ located over feed conveyor 316. For example, a spectral imaging system may be utilized between grinding mill 320 and proportioning station 330, and/or between clinker cooler 360 and finish grinding mill 380, or elsewhere in cement manufacturing plant 300.

Turning now to FIGS. 4-8, an arrangement of spectral imaging system 400 or 400′ will be described. With reference to FIG. 4, spectral imaging system 400 may include a frame 410 that supports an spectral imaging camera 420 and illumination sources 430 of spectral imaging system 400 over a conveyor 440 of an earthen material processing facility that is transporting a stream or flow of earthen material 446, such as earthen material, relative to spectral imaging system 400 (i.e., earthen material 446 is continuously transported past spectral imaging camera 420). For example, in the mineral processing system 100 of FIG. 2, the conveyor 440 may be feed conveyor 130. In other examples, spectral imaging system 400 may be mounted over any other conveyor of mineral processing system 100 such as return conveyor 146, first transfer conveyor 148, second transfer conveyor 158, for example. In still other earthen material processing systems, spectral imaging system 400 may be mounted over a conveyor or elsewhere with a view of earthen material being transported by any other means of transport, or in any other location where earthen material is flowing or moving in the earthen material processing system or facility. In some examples, the spectral imaging system 400 may be mounted over, on, or within other equipment for conveying or transporting earthen material, such as over or in chutes, tubes, or tunnels; over or on haulage vehicles, such as dump trucks or other trucks, load-haul-dump loaders (LHD machines), front-end loaders, and the like; on or over drag conveyances or vibratory conveyances; or over a location where haulage vehicles of the earthen material processing system or mining operation must pass. In yet other examples, the spectral imaging system 400 may be mounted over a conveyor of a non-earthen material processing system, or over, in, or within another material processing component or material handling equipment that moves the material or in or on which the material is flowing.

spectral imaging system 400 may include one or more shrouds 450 mounted to frame 410 to shield the spectral imaging camera's field of view from direct illumination source from external illumination source sources (e.g., sunlight) other than illumination sources 430. Such shielding improves the consistency of illumination of earthen material 446 on conveyor 440 (primarily by illumination sources 430), by reducing variations in secondary illumination from external sources that may be caused by reflections and shadows from personnel and equipment moving outside of spectral imaging system 400 and other causes. Shrouds 450 also shield high-intensity illumination source from illumination sources 430 from being directed into the eyes of personnel who may be working along conveyor 440. When used with feed conveyor 130 of mineral processing system 100 (FIG. 2), spectral imaging system 400 may desirably be positioned within feed tunnel 124 to shield spectral imaging system 400 from direct sunlight to reduce variation in the illumination of earthen material on feed conveyor 130 as it passes beneath spectral imaging camera 420.

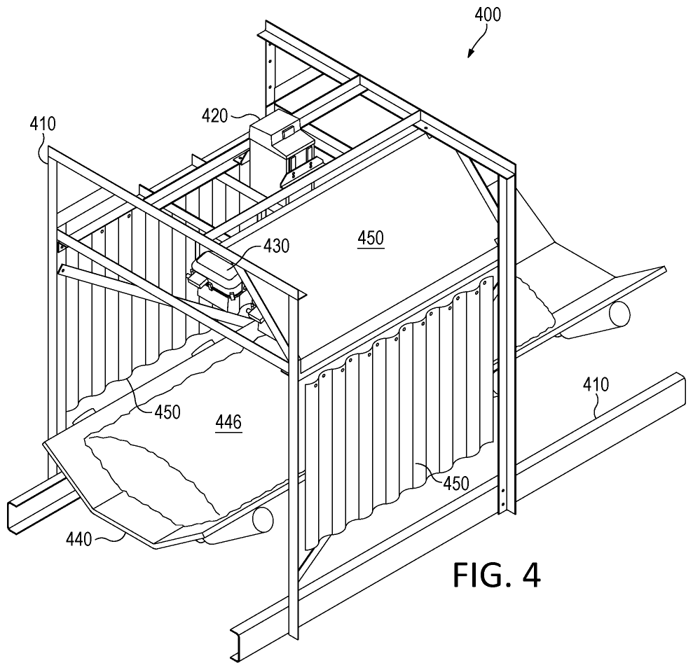

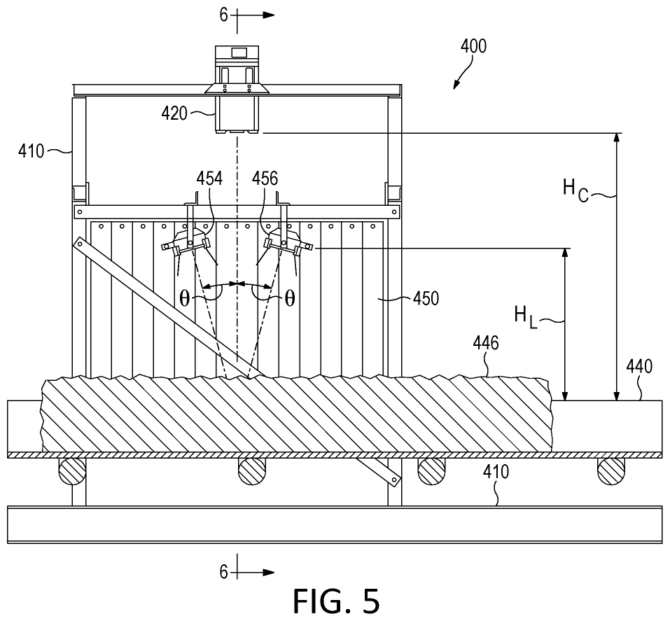

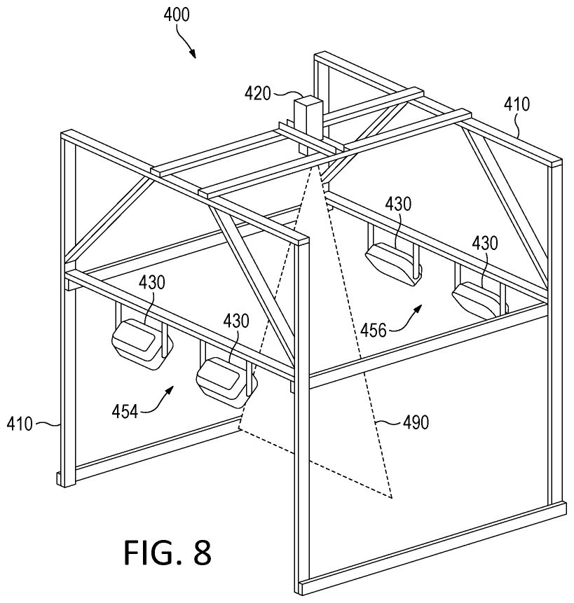

FIG. 5 illustrates that illumination sources 430 may be mounted relative to a field of view 490 (FIG. 8) of spectral imaging camera 420 both forwardly and aft along the direction of movement of conveyor 440. The illumination sources may further include optical lenses to substantially match the spectral imaging camera's field of view 490. A first bank 454 of the illumination sources 430 is spaced longitudinally forward relative to the field of view 490, and a second bank 456 of the illumination sources 430 is spaced aft relative to the field of view 490, with one or both the first and second banks 454, 456 optionally positioned lower than spectral imaging camera 420 and closer to earthen material 446 than camera 420. Each bank 454, 456 may be aimed at an angle θ of between 10 degrees and 70 degrees relative to the field of view 490 of spectral imaging camera 420 (wherein the field of view 490 is coincident with section line 6-6 in FIG. 5), or more preferably between 20 and 50 degrees or even more preferably between 40 and 50 degrees, or approximately 45 degrees, relative to the field of view 490. As illustrated in FIG. 6, each of the first and second banks of illumination sources 454, 456 may include multiple illumination source sources or illumination sourcing units spaced apart laterally across the width of the conveyor 440 or at the same position along the direction of movement of the conveyor. Each of the illumination sources 430 may include any of a variety of illumination sources, such as LEDs, halogen lamps, supercontinuum lasers, or other illumination source sources. The illumination source may be mounted to the spectral imager and not separate therefrom.

The spectral imaging camera 420 may be mounted at a height H_{c} between approximately 1 and 4 meters above conveyor 440, or between 1.5 and 2.5 meters above conveyor 440, or more preferably approximately 1.9 meters above conveyor 440, but other configurations are possible. The spectral imaging camera 420 may preferably have a depth of field in the range of about 0.2 meters to about 0.6 meters, or more, so that the upper surfaces of all earthen material 446 carried by conveyor 440 are in focus as the earthen material 446 passes through the field of view 490. The illumination sources 430 may be mounted at a height HL of between 0.4 and 0.8 meters above conveyor 440, or between 0.5 and 0.7 meters above conveyor 440, or more preferably approximately 0.6 meters above conveyor 440, but other configurations are possible. The illumination sources 430 may include highly polished curved or parabolic reflectors to focus or direct illumination source of substantially even, spatially-homogenous intensity on a target region overlapping the field of view 490 and spanning from the surface of the conveyor to a height above conveyor 440 inclusive of a maximum height of earthen material 446 on conveyor 440 and within the depth of field of spectral imaging camera 420. It may be desirable for the illumination sources 430 to be configured so that spectral irradiance reaching the spectral imaging camera 420 may exceed 0.04 watts per steradian per square meter (W/(sr·m²)), or more preferably more than 0.05 W/(sr·m²), or between 0.04 and 0.06 W/(sr·m²), averaged for all spectral bands captured by the spectral imaging camera 420, as determined based on illumination source reflected by a Lambertian surface that absorbs 50% of the illumination (e.g., a calibration panel).

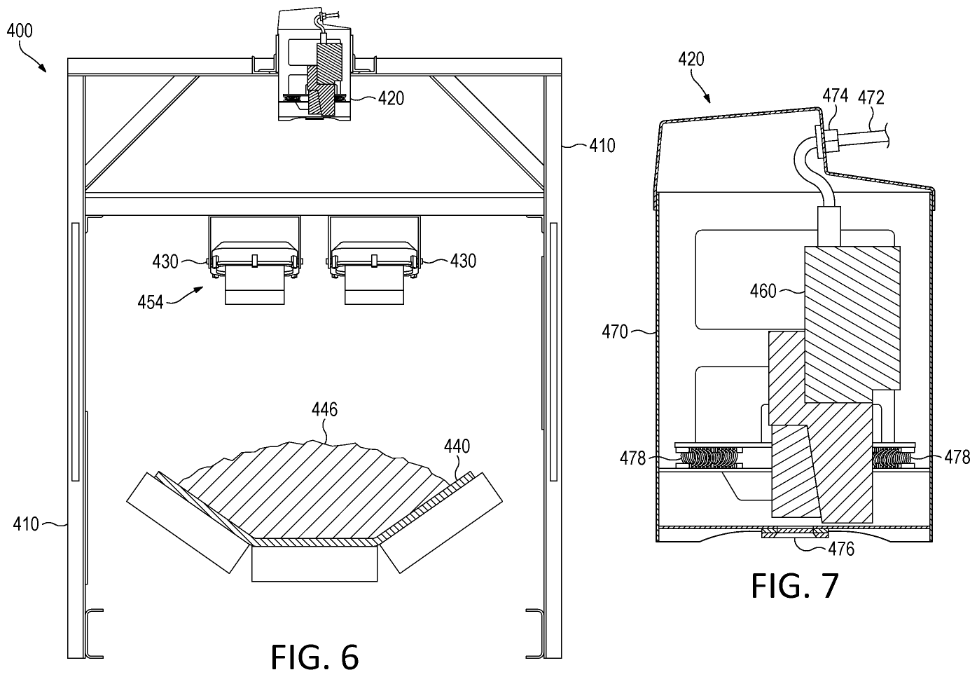

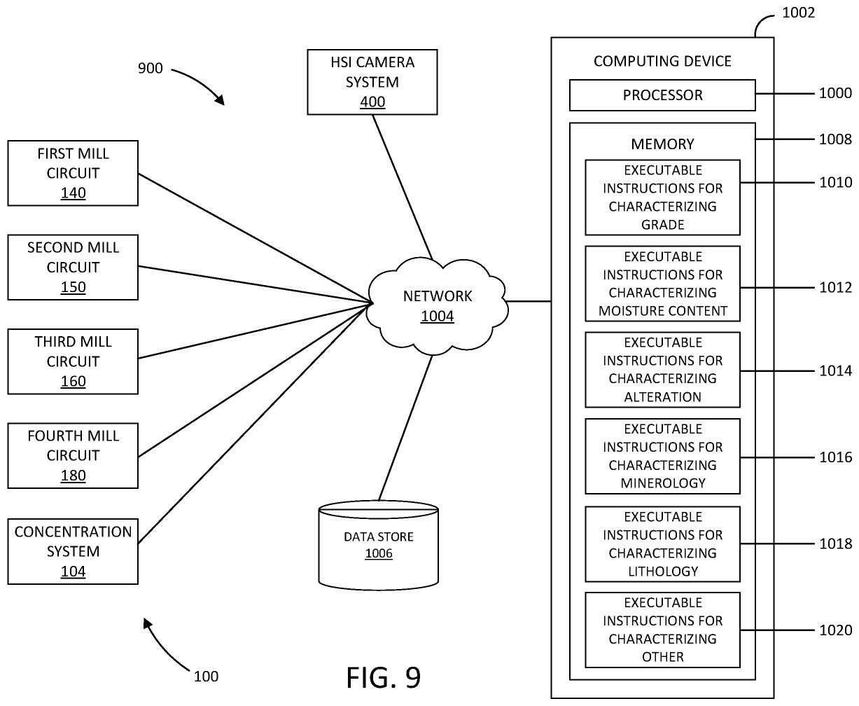

With reference to FIG. 7, spectral imaging camera 420 may include a spectral imager 460 mounted within a protective housing 470. The housing 470 may be hermetically sealed or sufficiently impervious to environmental dust and debris to inhibit damage to the sensitive electronics of the spectral imager 460. A cable 472 carrying data and/or power may exit housing 470 through an aperture that carries a seal, grommet, or O-ring 474 and may be connected to a processor 1000 (FIG. 2A), which is further described below with reference to FIG. 9. Housing 470 may include a window 476 of optically transparent material through which the objective lens of spectral imager 460 is aimed. Window 476 is preferably a flat plate formed of a synthetic fused silica that is OH free, sold by Heraeus Quartzglas GmbH & Co. KG under the trademark SUPRASIL. The OH-free synthetic fused silica from which window 476 is made may preferably have very high transmission (generally exceeding 90%) throughout the visible spectrum, near infrared (near IR) and short wavelength infrared spectrum and may preferably have minimal interactions with the electromagnetic spectrum from 950 nm to 2500 nm or more preferably from 400 nm to 2500 nm. Other optical materials having high transmission in the visible, near IR, and short wavelength IR spectrums may also be suitable for window 476. Spectral imager 460 may be supported on a shock-absorbing mount device or material 478 within housing 470, such as springs or resilient material, so that the shock-absorbing mount 478 is interposed between the housing 740 and spectral imager 460 to help isolate spectral imager 460 from external shocks and vibration. The housing 470 and mount 478 may also insulate the spectral imager 460 from changes in temperature, humidity, and other environmental influences that may impact its performance.

With reference to FIG. 8, the spectral imager 460 of spectral imaging camera 420 has a field of view 490 spanning the width of the conveyor 440 at the top of the conveyor 440. The field of view 490 of the spectral imaging imager 460 may have a lateral angular field of view of approximately 40 degrees, for example, or between 30 and 60 degrees, for example. The spectral imager 460 is preferably a line, set, batch, or pattern scanning imager or pushbroom imager that captures 288 bands (channels) of the electromagnetic spectrum in the range of approximately 950-2500 nm (or more preferably 400-2500 nm, or potentially 400-3000 nm). In one example, this can be along a single scan line, at a controllable acquisition rate of between approximately 1 Hz and 500 Hz (or higher), and with a spectral resolution in the range of approximately 4 nm to 10 nm, but other configurations are possible. The spectral imager 460 may have a spectral FWHM of less than 2 bands and a spatial FWHM of less than 2 pixels, and a peak signal to noise ratio greater than 7500 at full resolution. As an example, successive line scans or portions of line scans (or successive line captures in the case of a pushbroom imager) may be aggregated in a memory of the spectral imager 460 or in memory 1008 associated with processor 1000 to form a multi-dimensional data set representing a frame of a spectral image. In other examples, the spectral imager 460 may include a spectral scanning frame camera or snapshot frame imager having a larger longitudinal field of view than the line-scanning field of view 490 illustrated in FIG. 8.

In the example of line scanning spectral imager, the acquisition rate of the spectral imager 460 may be modulated based on the speed of the conveyor 440 to capture successive line scans, portions of line scans, or line captures, that can be aggregated in machine readable memory to form a spectral image or frame of spectral data for a spatial scene of the earthen material 446 being moved by the conveyor 440. Thus, the acquisition rate of the spectral imager 460 may be synchronized or adjusted to be synchronous with the movement of the conveyor 440 (e.g., conveyor belt movement), with a rate of transport of the earthen material 446 by the conveyor 440, or with the movement of another means of transport or conveyance of the earthen material. In this manner, the movement of the conveyor 440 establishes a longitudinal scan relative to the lateral scan line (or line capture) of the spectral imager 460. In other words, the scan line or line capture of the spectral imager 460 is scanned longitudinally along the earthen material 446 by virtue of movement of the conveyor 440 longitudinally relative to the spectral imager 460. If necessary, the spatial resolution of the spectral imager 460 may be reduced to increase the acquisition rate. The speed of the conveyor 440 may be determined from encoders in rotary components or by optical or electronic sensors associated with the conveyor belt, or by other means. Alternatively, the speed of the conveyor 440 may be consistent enough that the acquisition rate of spectral imager can be calibrated one time at setup to achieve accurate images of earthen material being transported by the conveyor 440, without the need for modulation of the acquisition rate. In some examples the illumination sources 430 may be strobed, for example if utilizing LEDs, and the strobe may be triggered by a signal from the spectral imager 460 and/or the strobe rate may be slaved to the acquisition rate of the spectral imager 460. In some examples, the intensity of the illumination sources 430 may be modulated based on the acquisition rate of the spectral imager 460 so as to ensure that sufficient reflected illumination reaches the spectral imager.

Turing now to FIG. 9, an earthen material characterization system 900 may be configured to utilize spectral imaging systems and methods to characterize one or more characteristics of earthen material and/or other earthen materials, such as ore grade, moisture content, mineral alteration, mineralogy, and lithology. The earthen material characterization system 900 is illustrated in use with a mineral processing system 100 for controlling one or more parameters or aspects of processing equipment or operations of mineral processing system 100, such as equipment of first, second, third and/or fourth mill circuits 140, 150, 160, and/or 180, and/or equipment of concentration system 104. In other examples, the earthen material characterization system 900 may be utilized with other types of earthen material processing systems, such as cement plant 300 (FIG. 3) or an adhesive manufacturing facility (not illustrated), to provide control inputs to milling equipment, dryers, and/or other kinds of earthen material processing and handling equipment. This and other arrangements and elements (e.g., machines, interfaces, function, orders, and groupings of functions, etc.) can be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by one or more components may be carried out by firmware, hardware, and/or software. For instance, and as described herein, various functions may be carried out by a digital processor executing instructions stored in memory.

Among other components, earthen material characterization system 900 may include spectral imaging system 400 which is communicatively coupled to a computing device 1002, which is in communication with a data store 1006. Computing device 1002 may include processor 1000, and memory 1008. Memory 1008 may store executable instructions for characterizing ore grade 1010, executable instructions for characterizing moisture content 1012, executable instructions for characterizing mineral alteration(s) 1014, executable instructions for characterizing mineralogy 1016, and/or executable instructions for characterizing lithology 1018.

The earthen material characterization system 900 shown in FIG. 9 is an example of one suitable system architecture for implementing certain aspects of the present disclosure. Additional, fewer, and/or alternative components may be used in other examples. It should be noted that implementations of the present disclosure are equally applicable to other types of devices and architectures. Any and all such variations, and any combinations thereof, are contemplated to be within the scope of implementations of the present disclosure.

Further, although illustrated as separate components, any number of components can be used to perform the functionality described herein. For example, although illustrated as being a part of computing device 1002, the components can be distributed via any number of devices (virtual or local). As one example, processor 1000 can be provided via one device, server, or cluster of servers, while memory 1008 may be provided via another device, server, or cluster of servers.

As shown in FIG. 9, computing device 1002, spectral imaging system 400, data store 1006, mill circuits 140, 150, 160, and 180, and concentration system 104 (and other elements of an earthen material processing system) may communicate with each other via network 1004, which may include, without limitation, one or more local area networks (LANs), wide area networks (WANs), and/or ad-hoc networks. Such networking environments are commonplace. Accordingly, network 1004 is not further described herein. It should be appreciated, however, that while computing device 1002, spectral imaging system 400, data store 1006, mill circuits 140, 150, 160, and 180, and concentration system 104 (and other elements of an earthen material processing system) may communicate with each other via network 1004 as illustrated in FIG. 9, other forms of communication are contemplated to be within the scope of this disclosure. For example, computing device 1002, spectral imaging system 400, and data store 1006 may communicate with each other via radio transmission systems, switching systems, data communication systems, satellite systems, fiber transmission systems, and wireline communication systems, among others. Computing device 1002 may also communicate with systems external to earthen material processing system 100, such as with software maintenance and licensing facilities—for example via an internet connection 1007 (FIG. 2A).

Any number of computing devices, spectral imaging systems, data stores, and/or mill circuits and other earthen material processing equipment may be utilized with the earthen material characterization system 900 within the scope of implementations of the present disclosure. Each may include a single device or multiple devices cooperating in a distributed environment. For instance, computing device 1002 could be provided by multiple server devices (e.g., edge computing or cloud computing servers) collectively providing the functionality of computing device 1002 as described herein. Additionally, other components not shown may also be included within the network environment.

Computing device 1002, spectral imaging system 400, mill circuits 140, 150, 160, and 180, and concentration system 104 (and other elements of an earthen material processing system) may have access (e.g., via network 1004) to at least one data store or repository, such as data store 1006, which may include any data related to characterization of earthen material characteristics, mineral processing, comminution and/or concentration operations, sizing and classification operations, dry milling operations, wet milling operations, separation and liberation methods, digital imaging and image-analysis methods, and spectral imaging techniques to inform excavation processes, as well as the metadata associated therewith. In some examples, data store 1006 may include any data and metadata related to training one or more machine learning models to predict one or more characteristics of earthen materials described herein, including one or more training data sets. In some examples, data store 1006 may include data related to ore grade information, moisture content information, mineral alteration information, mineralogy information, lithology information, and/or other relevant information, as well as any associated metadata. In some examples, data store 1006 may include data and metadata related to training sets associated with one or more of ore grade information, moisture content information, mineral alteration information, mineralogy information, lithology information, and other relevant information. In some examples, data store 1006 may include data collected via spectral imager 460. In some examples, data store 1006 may include spectral image data that may have been collected by one or more spectral imagers, and/or spectral image data that may have been previously collected.

In implementations of the present disclosure, data store 1006 is configured to be searchable for one or more of the data described above. Such information stored in data store 1006 may be accessible to any component of the earthen material characterization system 900. The content and volume of such information are not intended to limit the scope of aspects of the present technology in any way. Further, data store 1006 may be a single, independent component (as shown) or a plurality of storage devices, for instance, a database cluster, portions of which may reside in association with computing device 1002, spectral imaging system 400, another external computing device (not shown), and/or any combination thereof. Additionally, data store 1006 may include a plurality of unrelated data repositories or sources within the scope of examples of the present disclosure.

Data store 1006 may be local to computing device 1002 and spectral imaging system 400. In some examples, data store 1006 may be remote to computing device 1002 and spectral imaging system 400. Data store 1006 may be updated at any time, including an increase and/or decrease in the amount and/or types of data related to characterizing earthen material characteristics, mineral processing, comminution and/or concentration operations, sizing and classification operations, dry milling operations, wet milling operations, separation and liberation methods, digital imaging and image-analysis methods, and spectral imaging techniques to inform excavation processes, as well as the metadata associated therewith.

In some examples, the data collected by spectral imaging system 400 and stored in a data store, e.g., data store 1006, may be used to train one or more machine learning models (e.g., of computing device 1002) to measure, classify, and/or predict one or more characteristics of earthen material before and/or during the mineral processing process, such as in near real time and in-line with earthen material conveyance or other movement or operations happening as part of the earthen material processing system. The information on characteristics of the earthen material (or other earthen material handled by the earthen material processing system) can then be utilized by a control system or other components of the mineral processing facility to adjust parameters of the processing equipment or other operations of the facility.

Computing device 1002 may in some examples be integrated with or separate from the control systems and/or other components of the mineral processing facility. Computing device 1002 may in some examples be separate from spectral imaging system 400. In some examples, computing device 1002 may be implemented using one or more computers, servers, smart phones, smart devices, or tablets.

In some examples, computing device 1002 may be physically coupled to spectral imaging system 400 and/or one or more of the components of mineral processing system 100 (or other earthen material processing system), such as mill circuits 140, 150, 160, 180, classification system 170, and/or concentration system 104, but other configurations are possible. In other examples, computing device 1002 may not be physically coupled to spectral imaging system 400 and/or one or more of the components of mineral processing system 100 but co-located with the spectral imaging system 400 and/or one or more of the mill circuits 140, 150, 160, 180 and/or concentration system 104. In even further examples, computing device 1002 may neither be physically coupled to spectral imaging system 400 and/or one or more of the components of mineral processing system 100 nor co-located with the spectral imaging system and/or one or more of the mill circuits.

Computing devices, such as computing device 1002 described herein may include one or more processors, such as processor 1000. Any kind and/or number of processor may be present, including one or more central processing unit(s) (CPUs), graphics processing unit(s) (GPUs), other computer processors, mobile processors, digital signal processors (DSPs), microprocessors, computer chips, and/or data processing units configured to execute machine-language instructions and process data, such as executable instructions for characterizing ore grade 1010, executable instructions for characterizing moisture content 1012 executable instructions for characterizing mineral alteration(s) 1014, executable instructions for characterizing mineralogy 1016, executable instructions for characterizing lithology 1018.

Computing devices, such as computing device 1002, described herein may further include memory 1008. Any type or kind of memory may be present (e.g., read only memory (ROM), random access memory (RAM), solid state drive (SSD), and secure digital card (SD card). While a single box is depicted as memory 1008, any number of memory devices may be present. The memory 1008 may be in communication (e.g., electrically connected, etc.) to processor 1000. In some examples, memory 1008 is used to store data captured by the spectral imaging camera temporarily for characterization of earthen material. In some examples, the executable instructions of memory 1008 may be executed by processor 1000 to train one or more machine learning models to create a machine learned model for characterization of earthen material. As should be appreciated, one or more of the machine learned models as described herein may be embodied in and/or defined by one or more of the executable instructions described herein.

Memory 1008 may store executable instructions for execution by the processor 1000, such as executable instructions defining a machine learning model (or machine learned model) for characterizing ore grade 1010, executable instructions defining a machine learning model (or machine learned model) for characterizing moisture content 1012, executable instructions defining a machine learning model (or machine learned model) for characterizing mineral alteration 1014, executable instructions defining a machine learning model (or machine learned model) for characterizing mineralogy 1016, executable instructions defining a machine learning model (or machine learned model) for characterizing lithology 1018, and executable instructions defining a machine learning model (or machine learned model) for characterizing other material characteristics 1020. Processor 1000, being communicatively coupled to spectral imaging system 400 and mill circuits 140-180, and via the execution of executable instructions defining machine learning models for characterizing ore grade 1010, moisture content 1012, mineral alteration 1014, mineralogy 1016, lithology 1018, and other material characteristics 1020 may utilize spectral imaging data captured by spectral imaging system 400 (and/or other imaging systems) to train one or more machine learning models to transform the machine learning models into machine learned models for in-line material characterization and/or to control one or more parameters of the system and methods described herein for processing materials.

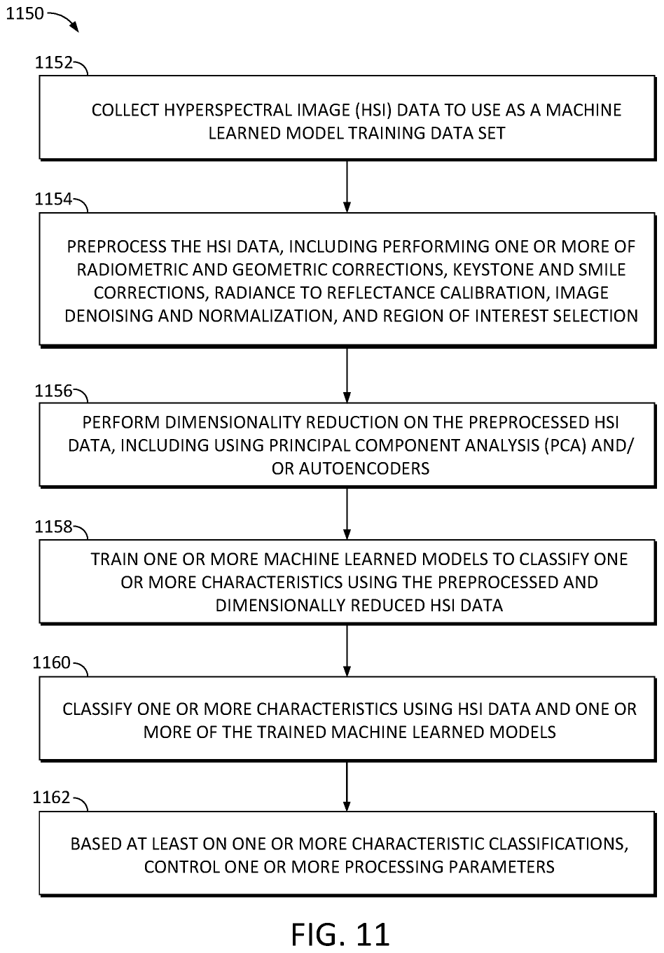

In operation, processor 1000 of computing device 1002 may execute executable instructions 1150 (FIG. 11) for characterizing ore grade 1010, to train a machine learning model (or further train a machine learned model) to classify an ore grade characteristic of earthen material. In some examples, the machine learning model or machine learned model may be trained using a training data set that may be collected separately from normal spectral imaging camera operation (step 1152 in FIG. 11). In some examples, to train a machine learned model to classify, measure, and/or predict an ore grade characteristic, the training data may undergo data preprocessing 1154 (FIG. 11). Data preprocessing 1154 may include, but is not limited to cleaning, organizing, etc. raw data to make it more suitable for building a training a machine learned model. Data preprocessing may enhance the quality of data to, in some examples, promote the extraction of meaningful insights from the data.

Processor 1000 of computing device 1002, executing executable instructions for characterizing ore grade 1010, may communicate with spectral imaging system 400 to preprocess the electromagnetic spectrum image data. In some examples, the preprocessing 1154 may include radiometric and/or geometric correction. In some examples, radiometric corrections are used to improve the radiometric quality of data, such as electromagnetic spectrum image data. Radiometric corrections (and/or calibrations) allow for the correcting of image reflectance, taking scene illumination and/or sensor influence into consideration. In some examples, geometric corrections attempt to correct for positional errors and to transform original image data into new image. In some examples, the radiometric and/or the geometric corrections occur at spectral imaging camera 420 and/or spectral imager 460 of spectral imaging system 400.

In some examples, the preprocessing 1154 may further include keystone and/or smile correction. As should be appreciated, smile and keystone are two types of optical aberrations that may impact the accuracy and the usability of spectral cameras. In examples, smile and keystone may appear as distortions of spectrum images. More specifically, smile generally refers to a spectral distortion and is primarily a property of a spectrograph, and can be seen as a spectral shift of the sensor over its entire field of view. Keystone generally refers to a spatial distortion, is mainly a property of a front objective, and can be seen as spatial misregistration of a spectrum. In some examples, the keystone and/or smile corrections occur at spectral imaging camera 420 and/or spectral imager 460 of spectral imaging system 400.

In some examples, the preprocessing 1154 may further include radiance to reflection calibration. As used herein, radiance is the amount of radiation coming from an area. Reflectance is the proportion of the radiation striking a surface to the radiation reflected off it. Some earthen materials can be identified by their reflectance spectra, correcting an image to reflectance may be a step toward locating or identifying features in an image. In some examples, for quantitative analysis of spectral image data, radiance images are corrected to reflectance images. In some examples, a white sample target may be used as a baseline for radiance calibration. In some examples, a flat white sample target or near perfect Lambertian surface may be advantageous to use because it may reflect illumination source at all angles equally, thereby creating nearly perfect diffuse reflectance.

In some examples, the preprocessing 1154 may further include image denoising and image normalization. In some examples, denoising may include reducing noise in image data. In some examples, noise apparent in image data may include additive Gaussian white noise, Poisson noise, salt-and-pepper noise, and/or other types of noise. In some examples, a denoising autoencoder may be used to perform denoising operations on spectral image data. In some examples, the denoising autoencoder may be based on the addition of noise to an input image to corrupt the data. This may be followed by image reconstruction. During the image reconstruction, the denoising autoencoder may learn the input features that may result in an overall improved extraction of latent representations.

In some examples, the preprocessing 1154 may further include region of interest selection. In some examples, based at least on the calibration of radiance to reflectance, a portion of the reflectance is low (e.g., in a shadowed region) (e.g., 10% reflectance). Here, the region of interest selection determines areas of low reflectance in the spectral image data and removes the data. In some examples, the removal of the data is done in real time (or near-real time). In some examples, the removal of the data occurs when the reflectance of certain pixels is below, meets, or exceeds a reflectance threshold. As should be appreciated, the size of the region of interest may be dynamic. Additionally, pixels can be masked depending on the ratio between two band indices, which could indicate the presence of a material (e.g., metal, cardboard, or the like) that the model is not trained on.

In some examples, the step of preprocessing 1154 may select a different contiguous region of interest when a determination is made that the reflectance of an already selected region of interest is low (e.g., at or below 10%). As should be appreciated, while a 10% reflectance threshold is described, additional and/or alternative reflectance thresholds may be utilized, such as, 5%, 12%, 15%, 20%, etc.

While five preprocessing steps are described herein, additional and/or fewer and/or alternative preprocessing steps to prepare the data for use in machine learning training and inference are contemplated to be within the scope of this disclosure. In some examples, one or more of the above preprocessing steps may be utilized. In some examples, a combination of one or more of the processing steps may be utilized. In some examples, none of the above preprocessing steps may be utilized. In some examples, no preprocessing steps may be utilized. In some examples, once the spectral data has been preprocessed, it may undergo dimensionality reduction as further described below.

To train a machine learning model to classify, measure, and/or predict a grade characteristic, processor 1000 of computing device 1002, executing executable instructions for characterizing ore grade 1010, may further perform dimensionality reduction 1156 (FIG. 11). As should be appreciated, machine learning algorithms face an issue called “curse of dimensionality,” which means as the size of the input data increases, a model may become more difficult to train. While this is not much of an issue for more traditional imaging systems, such as RGB imaging systems, spectral imaging systems face this issue because they may produce spectral image data with many more spectral channels (bands) of data—for example, 288 spectral bands, each in some examples representing a reflectance of a narrow band of only a few nanometers of wavelengths, such as between about 4 nm and 10 nm in width. Accordingly, one or more dimensionality reduction techniques may be used to reduce the feature space, obtain a stable and statistically sound machine learned model, and avoid the “curse of dimensionality” issue.

In some examples, processor 1000 of computing device 1002, executing executable instructions for characterizing ore grade 1010, may perform dimensionality reduction 1156 on the spectral image data via principal component analysis (PCA), discriminant projection analysis (e.g., linear DPA), or auto-encoders (e.g., a statistical method). In some examples, the statistical method may transform spectral image data into a new coordinate system where most of the variation in the data can still be described, however using lower coordinate dimensions. In some examples, PCA linearly transforms the spectral image data by projecting it onto a set of orthogonal axes, thereby maintain the variance of the spectral image data but reducing the data's overall dimensionality. In some examples, dimensionality reduction is fully supervised.

In some examples, processor 1000 of computing device 1002, executing executable instructions for characterizing ore grade 1010, may perform dimensionality reduction 1156 on the spectral image data utilizing autoencoders. In one example, an autoencoder may be an unsupervised artificial neural network that attempts to encode the data by compressing it into the lower dimensions (bottleneck layer or code) and then decoding the data to reconstruct the original input. The bottleneck layer or code holds the compressed representation of the input data. In another example, an autoencoder finds the representation of the data in a lower dimension by focusing on the important features by getting rid of noise and redundancy. In some examples, the data may be corrupted with noise or other anomalies before it is input to the autoencoder. One method for corrupting the input data with noise may utilize mathematical models for radiative transfer to simulate how the spectral image data would look like if the target area were to be lit by a diffuse illumination source. Such a simulation thus introduces noise for corrupting the spectral image data before it is input to the autoencoder. In some examples, an autoencoder is based on an encoder-decoder architecture, where the encoder encodes the high-dimensional data to lower-dimension and the decoder takes the lower-dimensional data and tries to reconstruct the original high-dimensional data. In some examples, utilizing autoencoders may be non-linear, based at least on the choice of activation function. In some examples, the use of autoencoders may train through gradient descent. In some examples, PCA may be used for smaller data sets, while autoencoders may be used for larger datasets, although either one may be used for any size dataset.

Processor 1000 of computing device 1002, executing executable instructions for characterizing ore grade 1010, may utilize the preprocessed and/or dimensionality reduced spectral image data to train a machine learning model to classify, measure, and/or predict an ore grade characteristic. In some examples, the machine learning model may include a three-dimensional convolutional neural network (3D CNN) that may be trained 1158 (FIG. 11) using the preprocessed and/or dimensionality reduced spectral image, but a two-dimensional CNN could also be used. In some examples, the preprocessed and dimensionally reduced spectral data (e.g., an image frame) may be used for the training 1158, such as a 7×7 patch of pixels. The entire image frame may be padded with zeros on the edges, allowing the model to process patches of pixels throughout the image frame without loss of information about pixels at the edge of the original image frame. As should be appreciated, alternatively sized pixel patches may be used (e.g., fed to the machine learned model), for example, during the training process 1158. The pixel patches preferably include an odd number of pixels along each spatial dimension of the patch, such as 3×3, 5×5, 7×7, 9×9, 11×11, 13×13, 15×15, etc. In some examples, the additional and/or alternative patch sizes selected may be based at least on the characteristic that the model is being trained to classify. For example, a 5×5 patch may be used when training a machine learned model to classify mineral alteration(s). In some examples, one or more of the patches of pixels is a portion of spectral image data obtained by, for example, the spectral imager 460. 3D CNNs may be utilized to analyze the spectral and spatial properties of a spectral image. In some examples, a center pixel of the patch is being classified, and the neighboring pixels are used to analyze spatial relationships within in the patch. Using patches of pixels larger than only a single pixel allows the model to analyze spatial properties (e.g., whether the pixel and its neighbors are homogenous, or the kinds of minerals and their distributions found within the patch), and such special properties may inform the determination of certain characterizations of earthen material, such as mineral alteration(s), lithology, etc. Depending on the geology and the spatial resolution of the camera, larger or smaller patch sizes might be more appropriate. Note that as patch size increases, the curse of dimensionality can reoccur, which is a practical limitation on the maximum possible patch size.

Using the training set of preprocessed, and dimensionally reduced spectral image data, processor 1000 of computing device 1002, executing executable instructions for characterizing ore grade 1010, may train the machine learning model (at step 1158) to transform the machine learning model into a machine learned model for characterizing an ore grade of earthen material (at step 1160). While a 3D CNN is described herein as an example of a machine learned model, in some cases, additional and/or alternative machine learned models that can classify may be used, such as two-dimensional (2D) CNNs, and the like. In some examples, the 3D CNN may include one or more convolution layers, certain kernel sizes, certain residual connections, and the like. In a desirable configuration, the 3D CNN may include six (6) layers, with kernel sizes ranging from 3×3×3 to 1×1×2, and one residual connection, but other configurations are possible. However, and as should be appreciated, in other examples other combinations of layers, kernel sizing, and residual connections may be used with in-line spectral imaging for characterizing the ore grade of earthen material moving through an earthen material processing facility. Additional layers may help with analysis of more complex data, and additional residual connections may be appropriate as the number of layers is increased. The particular network configuration may depend on the complexity of the information that the machine learning model is trying to extract in order to make an appropriate classification. The complexity of extracted information may vary from geology to geology. Accordingly, the network configuration may be determined for a particular mine or geology through hyperparameter optimization, wherein the parameter space is searched while iteratively altering the number of layers, the size of kernel, particular architectures, etc., and/or utilizing ablation studies, to find the best and/or most compact network for achieving the desired results.

The computing device 1002 including processor 1000 may use an ore grade characteristic machine learned model to determine (e.g., classify, measure, predict) a class label for an ore grade characteristic based at least on being trained on the spectral image data training set. As one example, after being trained, the ore grade characteristic machine learned model may classify a sample of spectral imaging data collected for newly mined earthen material being processed by the mineral processing system 100. For example, the class label may characterize an ore grade on a scale (e.g., a scale of 1 to 10, with 10 being high mineral content percentage) or on a volumetric percentage scale, using predetermined bands of predicted volumetric percentage between the lowest and highest possible amounts (e.g., Band 1=1% to 1.5% mineral content by volume, Band 2=1.5% to 2% mineral content by volume, etc.). Alternatively, the class label may characterize the ore grade more roughly, as ‘high-quality’, ‘medium-quality’, or ‘low-quality’, for example.

In some examples, the machine learned model may be trained on premise, and prior to classification for real time mineral processing. In some examples, the machine learned model may be regularly trained using a frequently-updated training set based on newly collected spectral image data and corresponding laboratory test data on earthen material samples collected (e.g., diverted from the conveyor after imaging) in near real time in synchronization with the spectral imaging data collection, with the laboratory test results later being matched with the corresponding spectral imaging data and fed to the machine learned model for feedback/training purposes. In some examples, the machine learned model may be trained using training feedback provided over a network and/or in the cloud.

Turning now to moisture content, in operation, to train a machine learning model to classify, measure, and/or predict a moisture content characteristic, processor 1000 of computing device 1002, executing executable instructions for characterizing moisture content 1012, may utilize the preprocessing, dimensionality reduction, and 3D CNN training steps discussed above with respect to training an ore grade characteristic machine learned model. Once trained, the computing device 1002 including processor 1000 may use a moisture content characteristic machine learned model to determine (e.g., classify, measure, or predict) a class label for a moisture content characteristic based on least on being trained on the spectral image data training set. As one example, after being trained, the moisture content characteristic machine learned model may classify a sample of newly mined earthen material based on spectral image data collected as the earthen material is being processed by the mineral processing system 100. In some examples, the class label may represent a percentage of moisture content by weight, or within pre-defined bands of moisture content by weight. In one example, the classification may be one of 0% moisture by weight, 1% moisture by weight, 2% moisture by weight, 3% moisture by weight, 4% moisture by weight, etc., for example. Alternatively, each of the possible classifications or class labels may represent an estimated range of weight percentages of moisture, or a median weight percentage+/−a tolerance (e.g., +/−1%, or +/−0.01%, or another tolerance in an amount therebetween), such that the possible classifications represent adjacent ranges/bands of moisture content from the lowest possible to highest possible moisture content.

Turing now to mineral alteration, in operation, to train a machine learning model to classify, measure, and/or predict a mineral alteration characteristic, processor 1000 of computing device 1002, executing executable instructions for characterizing mineral alteration(s) 1014, may utilize the preprocessing, dimensionality reduction, and 3D CNN training steps discussed above with respect to training an ore grade characteristic machine learned model. Once trained, the computing device 1002 including processor 1000 may use a mineral alteration characteristic machine learned model to determine (e.g., classify, measure, or predict) a class label for a mineral alteration characteristic based on least on being trained on the spectral image data training set. As one example, after being trained, the mineral alteration characteristic machine learned model may classify a sample of newly mined spectral image data. In some examples, the possible classifications or class labels may be one or more of oxidation, hydration, dehydration, kaolinization, epidotization, chloritization, sericitation, shock induced alteration, radioactive decay, serpentinization, dolomitization, pyritization, alkali alteration, pottasic alteration, phyllic alteration, k-feldspar alteration, quartz alteration and/or opalization, for example.

Turing now to mineralogy, in operation, to train a machine learning model to classify, measure, and/or predict a mineralogy characteristic, processor 1000 of computing device 1002, executing executable instructions for characterizing mineralogy 1016, may utilize the preprocessing, dimensionality reduction, and 3D CNN training steps discussed above with respect to training an ore grade characteristic machine learned model. Once trained, the computing device 1002 including processor 1000 may use a mineralogy characteristic machine learned model to determine (e.g., classify, measure, or predict) a class label for a mineralogy characteristic based on least on being trained on the spectral image data training set. As one example, after being trained, the mineralogy characteristic machine learned model may classify a sample of newly mined spectral image data. In one example, the class label may be selected from a set of any of various common trace minerals, mineral mixtures, and/or ranges of trace mineral content.

Turing now to lithology, in operation, to train a machine learning model to classify, measure, and/or predict a lithology characteristic, processor 1000 of computing device 1002, executing executable instructions for characterizing lithology 1018, may utilize the preprocessing, dimensionality reduction, and 3D CNN training steps discussed above with respect to training an ore grade characteristic machine learned model. Once trained, the computing device 1002 including processor 1000 may use a lithology characteristic machine learned model to determine (e.g., classify, measure, or predict) a class label for a lithology characteristic based on least on being trained on the spectral image data training set. As one example, after being trained, the lithology characteristic machine learned model may classify a sample of newly mined spectral image data. In one example, the class label may be selected from granodiorite, diorite, shale, and sandstone.

While various earthen material characteristics such as ore grade, moisture content, mineralogy, mineral alteration, and lithology are described herein, systems and methods described herein may train additional and/or alternative machine learned models to classify, measure, and/or predict other earthen material characteristics and/or characteristics of non-earthen materials.

Once trained, the machine learned models may, using processor 1000 of computing device 1002, or another computing device or control system, be used to control certain equipment or processes within the earthen material processing facility (step 1162 in FIG. 11). As one example, if an ore grade characteristic machine learned model classifies newly mined spectral image data as high-quality grade, then processor 1000 of computing device 1002 may send a signal to one or more of the mill circuits 140, 150, 160, and 180, and/or equipment of concentration system 104, and/or to the control systems for such mill circuits or concentration systems (or other earthen material processing systems), to impact the operation of such systems, or change one or more processing parameters related to ore grade, based at least on the prediction, classification, and/or measurement of an earthen material characteristic from one or more of the machine learned models. The signal could be a control signal or could be the characteristic derived from the earthen material characterization system 900, which the control system of the processing system may take into account to adjust certain operations in order to reduce waste and energy usage. The control system may utilize rules stored in a rules repository (e.g., in data store 1006 or elsewhere), to determine, based on the particular class label determined by the machine learned model, the control signals that should be sent to one or more equipment or processes of mineral processing system. The rules may further include linear or non-linear control algorithms or methodologies to avoid changing process parameters more quickly than needed, or otherwise introducing unwanted perturbations in the mineral processing system. Feedback from sensors in the earthen material processing system may also be utilized for closed-loop control in addition to process parameters (setpoints) that may be changed in response to signals transmitted from spectral imaging system based on the classification or class labels determined by processor 1000.

In some examples, the parameters of the mineral processing equipment that may be adjusted or controlled based on the ore grade, mineralogy, lithology, moisture content, and/or mineral alteration(s) of earthen material being fed into the mineral processing system (as classified by the earthen material characterization system 900) may include the mix of earthen material drawn from different stockpiles 120 through one or more chutes onto one or more conveyors leading to the feed conveyor 130 (FIG. 2) with which the spectral imaging system 400 is utilized in-line. In another example, a sorting system located in-line with a feed conveyor, or downstream thereof, may be manually or automatically controlled, based on classifications of ore grade, mineral alteration, moisture content, lithology and/or mineralogy obtained from the in-line spectral imaging data and machine learned models of the earthen material characterization system 900, to remove earthen material having a low ore grade, unwanted mineralogy or lithology, or other deleterious content (such as equipment parts). In examples wherein the spectral imaging system 400 is used to analyze earthen materials transported by a haulage vehicle, as previously described above, the earthen material characterization system 900 may be used to communicate sorting instructions to the haulage vehicle or to a driver of the haulage vehicle upon detecting that the earthen material has a low ore grade, unwanted mineralogy or lithology, or other deleterious content; and in response to the sorting instructions the haulage vehicle's load can be segregated, delivered to a different stockpile than usual, or excluded from processing altogether. In examples wherein the spectral imaging camera 400 is used to analyze earthen material transported by a conveyor, as previously described above, the earthen material characterization system 900 may be used to communicate sorting instructions to separate lifts for producing multiple stockpiles or heaps based on crucial characteristics such as presence of acid-consuming gangue mineralogy, clay-rich composition, and copper mineralogy, from which material is sourced and placed on a specific heap in a manner or sequence that reduces the negative impact on the heap leach performance by aligning similar characteristics of earthen material together in the same heap.

In other examples, a rate of dispensing of chemicals (such as limestone or other pH controlling chemicals) or grinding media that are added in SAG mill 162 and/or ball mill 182 may be manually or automatically controlled, using rules-based control algorithms, based on the classifications of ore grade, mineralogy, lithology, moisture content and/or mineral alteration(s) of earthen material determined from the in-line spectral imaging data and machine learned models of the earthen material characterization system 900, to thereby increase the quantity or quality of output, energy usage, or other performance characteristics of such milling equipment or of the overall mineral processing facility. The rotational speed or other parameters of SAG mill 162 and/or ball mill 182 may also be adjusted manually or automatically by rules-based control methods in response to classification of the ore grade, mineralogy, and/or mineral alteration(s) of the earthen material determined using in-line spectral imaging data and machine learned models of the earthen material characterization system 900. In still other examples, a rate of dispensing of reagents (such as collectors, frothers, pH modifiers, activators, and depressants) into one or more froth floatation machines 190 of concentration system 104, and/or other parameters of froth floatation machines 190 (such as air flow, mechanical agitation rate, etc.), may be manually or automatically controlled, using rules-based control algorithms, based on classifications of ore grade, mineralogy, and/or mineral alteration(s) obtained from the in-line spectral imaging data and machine learned models of the earthen material characterization system 900. For example, in response to a mineralogy classification by the earthen material characterization system 900 indicating an increase in phyllosilicates, a reagent usage rate can be increased (e.g., to a predefined value based on prior experience or experimentation) to thereby avoid a reduction in copper recovery (and increased loss of copper to tailings) that can otherwise result from such an increase in phyllosilicates. Classifications of ore grade, mineralogy, and/or mineral alteration(s) obtained using the in-line spectral imaging data and machine learned models of the earthen material characterization system 900 may also be used for mines extraction planning (i.e., which regions of earthen material should be blasted or excavated for feeding to the stockpile 120 of mineral processing facility 100), or for making manual changes to the parameters of mineral processing equipment.

In some geologies, the mineral alteration and/or lithology of the earthen material may be indicative of breakage properties of the earthen material, such as its bond work index, which is a measure of the resistance to breakage in crushing and grinding. In response to a predicted increase in the bond work index, as indicated by classifications of mineral alteration and/or lithology determined by the earthen material characterization system 900, certain milling processes can be more dynamically controlled. For example, operational parameters of the SAG mill 162 may be controlled based on rules-based control algorithms to provide increased throughput, decreased power usage, and/or improved size reduction.

In some examples, the machine learned models of the earthen material classification system 900 may classify the moisture content of a sample of newly mined earthen material based on spectral image data collected in-line as the earthen material is being processed by the mineral processing system 100. Upon classification of the moisture content, a processor 1000 of computing device 1002 may send a signal to one or more of the mill circuits 140, 150, 160, and 180, and/or equipment of concentration system 104, and/or to the control systems for such mill circuits or concentration systems (or other earthen material processing systems), to impact the operation of such systems, or change one or more processing parameters related to moisture content. For example, an amount or flow rate of dilution water 161 added to SAG mill 162 and/or ball mill 182 may be adjusted or controlled manually or automatically by rules-based control methods in response to classification of the moisture content of the earthen material determined using spectral imaging data and machine learned models of earthen material characterization system 900. In some examples, a flow rate of water that is sprayed onto the rolls of HPGR machine 152 of second mill circuit 150, or that is otherwise added to second mill circuit 150, may be adjusted or controlled manually or automatically by rules-based control methods in response to the classification of the moisture content of the earthen material, as determined using in-line spectral imaging data and machine learned models of earthen material characterization system 900. Alternatively, a rate of dewatering in SAG mill 162, ball mill 182, and/or HPGR machine 152 may be adjusted or controlled manually or automatically by the rules-based control methods in response to the classification of the moisture content. Controlling the water spray, flow rate, addition rate, or dewatering in this manner may help to avoid clumping of earthen material in HPGR machine 152 or may improve wear or equipment performance. The transfer of earthen material through and between dry milling circuits (such as first and second mill circuits 140 and 150) may generate excessive dust that must be reduced for safety reasons or for compliance with government regulation. Dust reduction may be accomplished by applying a water mist to earthen material as it is being conveyed by a conveyor, or by other dust reduction techniques, However, excessive misting can lead to decreased efficacy of the dry milling processes. Accordingly, the moisture content determined using in-line spectral imaging data can be used to control the pressure and/or flow rate of water delivered to one or more misting nozzles to thereby increase or decrease the volume of water applied per metric ton of earthen material. By reducing downstream variation in the moisture content of earthen material and minerals, such control techniques may also aid in improved filtering, faster dewatering, decreased drying, and/or improved pelletizing of mineral concentrate, and/or more accurate production accounting, wherein the weight of earthen material or extracted minerals are measured for production accounting purposes. Such control techniques may also reduce dust, transportation costs, and/or handling problems that can arise from poor control of moisture content.

In some examples, control of the mill circuits 140, 150, 160, 180 (and/or other controllable components of the mineral processing facility 100 of FIG. 2A, such as a comminution facility 102 and/or concentration facility 104, among others) may be centrally managed. In other examples, control of the mill circuits 140, 150, 160, 180 (and/or other controllable components of the comminution facility 102 and/or concentration facility 104) may be managed in a distributed manner. In some examples, processor 1000 of computing device 1002 may alter and/or change one or more parameters of one or more of the mill circuits 140, 150, 160, 180, and/or other components of mineral processing facility 100. In some examples, processor 1000 of computing device 1002 may send a signal to a centrally managed location and/or to a user of (or within) mineral processing facility 100 (e.g., such as a shovel operator, mine foreperson, etc.), who may alter and/or change one or more parameters of one or more of the mill circuits 140, 150, 160, 180, and/or other components of mineral processing facility 100.

In some examples, spectral imaging systems 400 may be used in multiple locations in an earthen material processing facility at various stages of processing, and the resulting earthen material characterizations (classifications) of ore grade, mineralogy, lithology, mineral alteration and/or moisture content from the multiple systems may be compared, in processor 1000 or elsewhere, to identify underperforming equipment or equipment malfunctions. The system may trigger alarms or send alert messages to on-duty personnel or on-call personnel when such underperformance or errors are detected.

Similarly, a spectral imaging system 400′ may be utilized in a cement manufacturing plant 300 or adhesives manufacturing facility to characterize or classify one or more earthen material characteristics of earthen raw materials in-line and during processing of earthen materials. Parameters of equipment in cement manufacturing plant 300 may be adjusted or controlled based on the ore grade, mineralogy, mineral alteration(s), lithology, moisture content or other characteristics of the limestone and/or other earthen raw materials being utilized (as classified by the earthen material characterization system 900). Such controllable parameters may include the mix of earthen materials drawn from different stockpiles 312, grinding and regrind rates of grinding mill 320 and/or finish grinding mill 380, proportioning amounts of clay and sand metered by proportioning station 330, rate and residency parameters of the meal grinding mill (not illustrated), a preheating temperature of preheating tower 340, residency time in kiln 350, rates of dispensing of gypsum and/or other additives at blending station 370, or the grinding and/or regrind rates or other parameters of finish grinding mill 380. The various parameters of such equipment and/or processes of cement manufacturing plant 300 may be manually or automatically controlled, using rules-based control algorithms, based on the classifications of ore grade, mineralogy, mineral alteration(s), lithology, moisture content and/or other characteristics of the limestone and/or other earthen raw materials determined from the in-line spectral imaging data and the machine learned models of earthen material characterization system 900, to thereby increase output of such equipment or of the overall cement manufacturing plant 300, improve the quality of the cement produced, reduce waste, reduce carbon footprint, and/or reduce energy consumption.

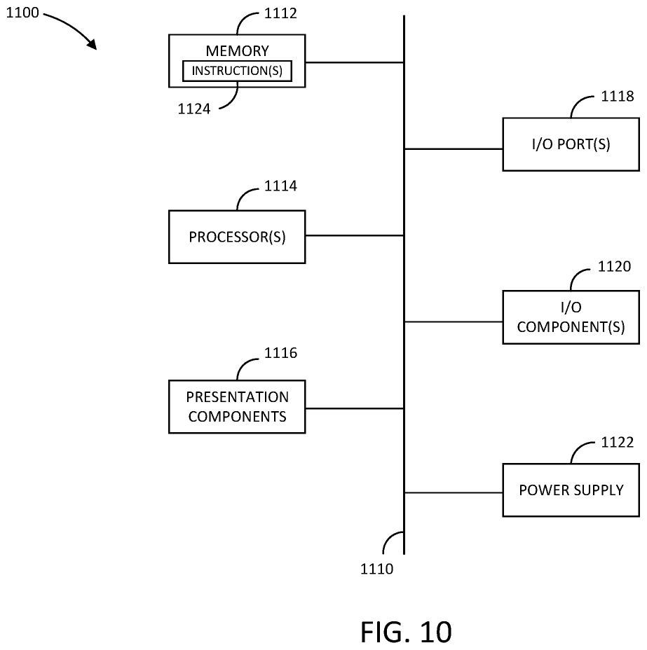

Having described an overview of examples of the present disclosure, an exemplary operating environment in which examples of the present disclosure, such as one or more components of the earthen material characterization system 900, may be implemented is described below in order to provide a general context for various aspects of the present disclosure. Referring now to FIG. 10 in particular, an exemplary operating environment for implementing examples of the present disclosure is shown and designated generally as computing device 1002. Computing device 1002 is but one example of a suitable computing environment and is not intended to suggest any limitation as to the scope of use or functionality of the disclosure. Neither should computing device 1002 be interpreted as having any dependency or requirement relating to any one or combination of components illustrated.

The examples may be described in the general context of computer code or machine-useable instructions, including computer-executable instructions such as program modules, being executed by a computer or other machine, such as a cellular telephone, personal data assistant or other handheld device. Generally, program modules including routines, programs, objects, components, data structures, etc., refer to code that perform particular tasks or implement particular abstract data types. The methods and systems according to the present disclosure may be practiced in a variety of system configurations, including hand-held devices, consumer electronics, general-purpose computers, more specialty computing devices, etc. The methods and systems according to the present disclosure may also be practiced in distributed computing environments where tasks are performed by remote-processing devices that are linked through a communications network.

With reference to FIG. 10, computing device 1002 may include a bus 1110 that directly or indirectly couples the following devices: memory 1112, one or more processors 1114, one or more presentation components 1116, input/output (I/O) port(s) 1118, input/output component(s) 1120, and illustrative power supply 1122. Bus 1110 represents what may be one or more busses (such as an address bus, data bus, or combination thereof). Although the various blocks of FIG. 10 are shown with lines for the sake of clarity, in reality, delineating various components is not so clear, and metaphorically, the lines would more accurately be grey and fuzzy. For example, one may consider a presentation component such as a display device to be an I/O component. Also, processors have memory. Skilled persons will recognize that such is the nature of the art and will appreciate that the diagram of FIG. 10 is merely illustrative of an exemplary computing device that can be used in connection with one or more examples. Distinction is not made between such categories as “workstation,” “server,” “laptop,” “hand-held device,” etc., as all are contemplated within the scope of FIG. 10 and reference to “computing device.”

Computing device 1002 typically may include a variety of computer-readable media. Computer-readable media can be any available media that can be accessed by computing device 1002 and may include both volatile and nonvolatile media, and removable and non-removable media. By way of example, and not limitation, computer-readable media may include computer storage media and communication media.

Computer storage media may include both volatile and nonvolatile, removable and non-removable media implemented in any method or technology for storage of information such as computer-readable instructions, non-transitory computer-readable media, data structures, program modules or other data. Computer storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 1002. Computer storage media does not include signals per se.

Communication media typically embodies computer-readable instructions, data structures, program modules or other data in a modulated data signal such as a carrier wave or other transport mechanism and may include any information delivery media. The term “modulated data signal” means a signal that has one or more of its characteristics set or changed in such a manner as to encode information in the signal. By way of example, and not limitation, communication media may include wired media such as a wired network or direct-wired connection, and wireless media such as acoustic, RF, infrared and other wireless media. Combinations of any of the above should also be included within the scope of computer-readable media.

Presentation components 1116 present data indications to a user or other device. Exemplary presentation components include a display device, speaker, printing component, vibrating component, etc.

I/O port(s) 1118 allow computing device 1002 to be logically coupled to other devices including I/O components 1120, some of which may be built in. In some instances, inputs may be transmitted to an appropriate network element for further processing.

The earthen material characterization system 900 may be further equipped with one or more other cameras, such as stereoscopic systems, infrared systems, or RGB systems and combinations of these, for aiding in the detection, recognition, classification, etc. as described herein.

Having identified various components in the present disclosure, it should be understood that any number components and arrangements may be employed to achieve the desired functionality within the scope of the present disclosure. For example, although some components are depicted as single components, many of the elements described herein may be implemented as discrete or distributed components or in conjunction with other components, and in any suitable combination and location. Some elements may be omitted altogether.

Moreover, various functions described herein as being performed by one or more entities may be carried out by hardware, firmware, and/or software, as described herein. For instance, various functions may be carried out by a processor executing instructions stored in memory. As such, other arrangements and elements (e.g., machines, interfaces, functions, orders, and groupings of functions, etc.) can be used in addition to or instead of those shown.

The subject matter of examples is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies.

Further, while examples of the present disclosure may generally refer to the systems and the schematics described herein, it is understood that the techniques described may be extended to other implementation contexts.

Examples disclosed herein are intended in all respects to be illustrative rather than restrictive. It will be obvious to those having skill in the art that many changes may be made to the details of the above-described examples without departing from the underlying principles of the disclosure.

The invention claimed is:

1. A system for characterizing earthen material in an earthen material processing system having at least one controllable operational parameter affecting processing of the earthen material, the system comprising:
a spectral imager positioned in view of earthen material moving within the earthen material processing system, the spectral imager configured to acquire spectral image data of a spatial scene of the earthen material;
a processor in communication with the spectral imager, the processor programmed with a machine learned model that is configured to process the spectral image data to determine an earthen material characteristic of the earthen material based on the spectral image data, the processor outputting a signal based on the earthen material characteristic so determined; and
the processor in communication with the earthen material processing system determining a recommendation to adjust the operational parameter in response to the signal.

2. The system of claim 1, wherein the spectral imager is configured to acquire the spectral image data while the earthen material is moving.

3. The system of claim 1, wherein the earthen material processing system includes a conveyor, and the spectral imager is mounted over the conveyor.

4. The system of claim 3, wherein the conveyor delivers a flow of earthen material to a comminution system of the earthen material processing system, the controllable operational parameter affecting operation of the comminution system.

5. The system of claim 1, wherein the spectral imager captures successive captures of spectral data that are aggregated by the spectral imager or the processor to form the spectral image data.

6. The system of claim 1, wherein the machine learned model includes a convolutional neural network.

7. The system of claim 1, wherein the processor is further programmed to preprocess the spectral image data to perform radiometric or geometric corrections prior to processing by the machine learned model.

8. The system of claim 1, wherein the processor is further programmed to perform dimensionality reduction on the spectral image data prior to processing by the machine learned model.

9. The system of claim 1, wherein the earthen material characteristic is a moisture content of the earthen material.

10. The system of claim 9, wherein the earthen material processing system includes at least one mill, and the operational parameter automatically adjusted in response to the signal includes a flow rate of water delivered to the mill and/or a dewatering rate of the mill.

11. The system of claim 9, wherein the earthen material processing system includes at least one dryer, and the operational parameter automatically adjusted in response to the signal includes an increased or decreased drying time within the dryer.

12. The system of claim 1, wherein the earthen material characteristic determined by the machine learned model is a mineral alteration of the earthen material.

13. The system of claim 12, wherein the earthen material processing system includes at least one mill, and the operational parameter automatically adjusted in response to the signal includes a grinding media volume of the mill, a rotational speed of the mill, a flow rate of water delivered to the mill, and/or a dewatering rate of the mill.

14. The system of claim 1, wherein:
the earthen material processing system includes a mineral processing system having a comminution system and a concentration system;
the spectral imager is located before or within the comminution system;
the earthen material characteristic determined by the machine learned model includes a classification of a mineral alteration of the earthen material; and
the operational parameter automatically adjusted in response to the signal includes a rate of addition of a reagent in the concentration system, the reagent being reactive with one or more desirable minerals in the earthen material.

15. The system of claim 1, wherein the spectral imager is located within a reclaim tunnel of the earthen material processing system.

16. The system of claim 1, further comprising one or more illumination sources positioned and oriented to direct illumination toward the earthen material for reflection by the earthen material to the spectral imager.

17. The system of claim 1, wherein the earthen material processing system includes at least one sorting system located in-line with a feed conveyor, or downstream thereof, and the operational parameter automatically adjusted in response to the signal includes the sorting system sorting to remove earthen material having a low ore grade or representing waste.

18. The system of claim 1, wherein the earthen material processing system includes at least one sorting system located in-line with a conveyor, or downstream thereof, and the operational parameter automatically adjusted in response to the signal includes the sorting system sorting material into different stockpiles based on a specific alteration and ore composition.

19. The system of claim 18, wherein the specific alteration includes clay composition and abundance, mineralogical composition of the gangue material, and/or mineralogical composition of copper minerals.

20. The system of claim 1, wherein the processor in communication with the earthen material processing system automatically adjusts the operational parameter.

21. The system of claim 1, wherein the spectral imager is mounted over a haulage truck route to scan a top surface of a haulage truck.

22. A method of operating an earthen material processing system, comprising:
acquiring a spectral image of earthen material moving within the earthen material processing system, the spectral image including spectral image data;
processing the spectral image data by a machine learned model operating on a data processor to determine an earthen material characteristic of the earthen material;
outputting a signal based on the earthen material characteristic determined by the machine learning model; and
determining a recommendation to adjust the operational parameter in response to the signal.

23. The method of claim 22, further comprising automatically adjusting an operational parameter of the earthen material processing system in response to the recommendation.
